(19) United States
(12) Patent Application Publication
(10) Pub. No.: US 2026/0182842 A1
(43) Pub. Date: Jul. 2, 2026

(54) SYSTEMS AND METHODS FOR ESTIMATING CORONARY ARTERY CALCIFICATION SCORES

(71) Applicant: iHealthScreen Inc., Queens Village, NY (US)

(72) Inventor: Alauddin BHUIYAN, Queens Village, NY (US)

(21) Appl. No.: 19/003,171

(22) Filed: Dec. 27, 2024

(51) Int. Cl. A61B 5/02; A61B 3/00; A61B 3/14; A61B 5/00; A61B 5/0205; G06T 7/00

(52) U.S. Cl. CPC A61B 5/02007; A61B 3/0025; A61B 3/14; A61B 5/0205; A61B 5/7275; G06T 7/0012; G06T 2207/20081; G06T 2207/20084; G06T 2207/30041

(57) ABSTRACT

A system for coronary artery calcium (CAC) estimation includes a processor and a memory, including instructions stored thereon, which when executed by the processor, cause the system to: extract a plurality of features from patient data using a first machine learning (ML) model, the patient data including a retinal image, sociodemographic data, and/or clinical data; refine the extracted plurality of features using an attention layer, the attention layer configured to highlight retinal structures with an indication of calcification risk; combine a subset of the plurality of features into a data vector using a second ML model; provide a CAC estimation based on the data vector; determine that the provided CAC estimation exceeds a predetermined threshold; and generate an output indicating a calcification risk level, based on the CAC estimation.

### GOVERNMENT SUPPORT

The subject matter of this disclosure was supported by the National Institutes of Health Small Business Innovation Research (NIH SBIR)—Grant No. 2R44EY031202-04A1.

### TECHNICAL FIELD

The present application relates to systems and methods for estimating calcification scores, and, more specifically, to a system and method for estimating coronary artery calcification scores using retinal imaging and multi-dimensional data integration.

### BACKGROUND

Cardiovascular disease (CVD) is one of the leading causes of death in the United States and many other countries worldwide. CVD includes a group of diseases that affect various parts of the heart and blood vessels. In general, CVD may impact all ages, genders, ethnicities, and/or socioeconomic backgrounds. Individuals may be born with CVD or develop CVD during any stage of their lives. Statistically, one in three women assigned female at birth dies from cardiovascular disease. One common type of CVD is coronary artery disease (CAD). CAD is generally caused by atherosclerosis, which is the buildup of atheromatous plaques within the walls of the coronary arteries potentially leading to a subsequent blockage, thereby limiting blood flow to the heart's muscles and preventing the heart muscle from receiving adequate oxygen. Calcification contributing to plaques frequently starts as micro-nodules (e.g., 0.5 to 15.0 μm), which eventually develop into larger calcium particles that form sheet-like structures (e.g., >3 mm) in the arteries.

As calcification progresses, coronary events may arise. For example, acute coronary events typically occur when a plaque ruptures leading to the formation of a thrombus. Furthermore, blockages of the coronary arteries can cause a heart attack or ischemic stroke. Coronary artery calcium (CAC) is a diagnostic marker, which may detect CVDs such as CAD. Current CAC tests, such as cardiac computed tomography (CT) scans, detect CAC directly but expose patients to radiation and/or may not be accessible for regular screenings.

Accordingly, there is a need for improved, minimally invasive CAC scoring, which enables secure, verifiable, and efficient data exchange between and across networks.

### SUMMARY

In accordance with aspects of the present disclosure, a system for coronary artery calcium (CAC) estimation includes a processor and a memory, including instructions stored thereon, which when executed by the processor, cause the system to: extract a plurality of features from patient data using a first machine learning (ML) model, the patient data including a retinal image, sociodemographic data, and/or clinical data; refine the extracted plurality of features using an attention layer, the attention layer configured to highlight retinal structures with an indication of calcification risk; combine a subset of the plurality of features into a data vector using a second ML model; provide a CAC estimation based on the data vector; determine that the provided CAC estimation exceeds a predetermined threshold; and generate an output indicating a calcification risk level, based on the CAC estimation.

In an aspect of the present disclosure, the first ML model may be a deep learning model.

In another aspect of the present disclosure, the sociodemographic data may include an age, gender, and/or race.

In yet another aspect of the present disclosure, the clinical data a presence of hypertension, cholesterol, blood pressure, body mass index (BMI), smoking status, and/or a presence of diabetes.

In a further aspect of the present disclosure, the instructions, when executed by the processor, may further cause the system to preprocess the patient data using pixel normalization, contrast enhancement, and/or image scaling.

In yet a further aspect of the present disclosure, the instructions, when executed by the processor, may further cause the system to preprocess the patient data using label encoding and/or one-hot encoding.

In an aspect of the present disclosure, the plurality of features extracted from the patient data may include a vessel width, focal arteriolar narrowing, arteriovenous nicking, central arteriolar light reflex, vessel branching patterns, hollenhorst plaque, papilledema, exudates, cotton wool spots, potential hemorrhages, and/or potential microaneurysms.

In another aspect of the present disclosure, the highlighted retinal structures may include an optic disc, a macula, and/or a retinal blood vessel.

In yet another aspect of the present disclosure, the attention layer may be implemented using self-attention, spatial attention, and/or channel-wise attention.

In a further aspect of the present disclosure, the second ML model may be a transformer-based fusion model configured to dynamically weigh the extracted plurality of features.

In accordance with aspects of the present disclosure, a processor-implemented method for coronary artery calcium (CAC) estimation includes: extracting a plurality of features from patient data using a first machine learning (ML) model, the patient data including at least one of a retinal image, sociodemographic data, or clinical data; refining the extracted plurality of features using an attention layer, the attention layer of the first ML model configured to highlight retinal structures with an indication of calcification risk; combining a subset of the plurality of features into a data vector using a second ML model; providing a CAC estimation based on the data vector; determining that the provided CAC estimation exceeds a predetermined threshold; and generating an output indicating a calcification risk level, based on the CAC estimation.

In an aspect of the present disclosure, the first ML model may be a deep learning model.

In another aspect of the present disclosure, the sociodemographic data may include an age, gender, and/or race.

In yet another aspect of the present disclosure, the clinical data a presence of hypertension, cholesterol, blood pressure, body mass index (BMI), smoking status, and/or a presence of diabetes.

In a further aspect of the present disclosure, the method further includes preprocessing the patient data using pixel normalization, contrast enhancement, and/or image scaling.

In yet a further aspect of the present disclosure, the method further includes preprocessing the patient data using label encoding and/or one-hot encoding.

In an aspect of the present disclosure, the plurality of features extracted from the patient data may include a vessel width, focal arteriolar narrowing, arteriovenous nicking, central arteriolar light reflex, vessel branching patterns, hollenhorst plaque, papilledema, exudates, cotton wool spots, potential hemorrhages, and/or potential microaneurysms.

In another aspect of the present disclosure, the highlighted retinal structures may include an optic disc, a macula, and/or a retinal blood vessel.

In yet another aspect of the present disclosure, the attention layer may be implemented using self-attention, spatial attention, and/or channel-wise attention.

In a further aspect of the present disclosure, the second ML model may be a transformer-based fusion model configured to dynamically weigh the extracted plurality of features.

In accordance with aspects of the present disclosure, a non-transitory computer readable storage medium including instructions that, when executed by a computer, cause the computer to perform a method for coronary artery calcium (CAC) estimation, the method including: extracting a plurality of features from patient data using a first machine learning (ML) model, the patient data including at least one of a retinal image, sociodemographic data, or clinical data; refining the extracted plurality of features using an attention layer, the attention layer of the first ML model configured to highlight retinal structures with an indication of calcification risk; combining a subset of the plurality of features into a data vector using a second ML model; providing a CAC estimation based on the data vector; determining that the provided CAC estimation exceeds a predetermined threshold; and generating an output indicating a calcification risk level, based on the CAC estimation.

### BRIEF DESCRIPTION OF THE DRAWINGS

A better understanding of the features and advantages of the disclosed technology will be obtained by reference to the following detailed description that sets forth illustrative embodiments, in which the principles of the technology are utilized, and the accompanying drawings of which:

FIG. 1 is an illustration of an exemplary coronary artery calcification (CAC) estimation system, in accordance with aspects of the present disclosure;

FIG. 2 is a block diagram of example components of a controller within the CAC estimation system of FIG. 1, in accordance with aspects of the present disclosure;

FIG. 3 is a block diagram of a data collection module of the CAC estimation system of FIG. 1, in accordance with aspects of the present disclosure;

FIG. 4 is a block diagram of a preprocessing module of the CAC estimation system of FIG. 1, in accordance with aspects of the present disclosure;

FIG. 5 is a block diagram of a feature extraction module of the CAC estimation system of FIG. 1, in accordance with aspects of the present disclosure;

FIG. 6 is a block diagram of a data fusion module of the CAC estimation system of FIG. 1, in accordance with aspects of the present disclosure;

FIG. 7 is a block diagram of a risk scoring module of the CAC estimation system of FIG. 1, in accordance with aspects of the present disclosure;

FIG. 8 is a block diagram of an explainability module of the CAC estimation system of FIG. 1, in accordance with aspects of the present disclosure;

FIG. 9 is a block diagram of a risk reporting module of the CAC estimation system of FIG. 1, in accordance with aspects of the present disclosure;

FIG. 10 is a block diagram illustrating data flow within the CAC estimation system of FIG. 1, in accordance with aspects of the present disclosure;

FIG. 11 is a flow diagram of an exemplary use of the CAC estimation system of FIG. 1, in accordance with aspects of the present disclosure; and

FIG. 12 is an exemplary illustration of a reported generated by the CAC estimation system of FIG. 1, in accordance with aspects of the present disclosure.

### DETAILED DESCRIPTION

The present application relates to systems and methods for estimating coronary artery calcium (CAC) scores using retinal imaging and multi-dimensional data integration.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Various alterations, rearrangements, substitutions, and modifications of the features illustrated herein, and any additional applications of the principles of the present disclosure as illustrated herein, which would occur to one skilled in the relevant art and having possession of this disclosure, are to be considered within the scope of the present disclosure.

Performing CAC score screening (e.g., a coronary calcium scan) is a non-invasive, cost-effective technique for testing the coronary arteries. A CAC score evaluates the severity of the plaques. Plaques are formed from various substances present in the blood such as fat, cholesterol, and/or calcium. Moreover, the formation of plaques can be calcified (e.g., hard), fatty/uncalcified (e.g., soft), and/or mixed. Soft plaques, also called fatty or vulnerable plaques, are caused by inflammation and may be hidden within the arterial walls, contributing to arterial narrowing and/or increasing the risk of rupture and sudden heart attacks. Calcified or hard plaques are generally more stable and less prone to rupture, suggesting that the presence of calcified plaque is a strong indicator of cardiovascular disease (CVD).

A CAC score may be calculated using a cardiac computed tomography (CT) scan, which can measure the amount of plaque and/or calcium present in the coronary arteries. The resulting CAC score is typically generated as a number, such as an Agatston score, which may be used by clinicians for further diagnosis. For example, the CAC score obtained from the CT scan may be used to assess the risk of CVD. While cardiac CT scans can easily detect calcified plaques, cardiac CT scan cannot visualize soft plaques without iodinated contrast, which would obscure calcium and thus affect scoring. However, the amount of calcified plaque often reflects the overall plaque burden and, therefore, a significant presence of calcified plaque may suggest a substantial amount of soft plaque. Further, cardiac CT scans may not be accessible for regular screenings. Even if CT scans were more readily accessible, such scans still expose patients to radiation, and thus would not be feasible for frequent testing.

The Agatston score is the standard scoring system for CAC scoring. After a radiologist analyzes and interprets CAC images, their team may send an official report to a doctor who initially ordered the screening. Grading for a CAC score screening is as follows: (0) no plaque is present, e.g., the risk of a heart attack is low; (1-10) a small amount of plaque is present, e.g., there is a less than a 10% chance of developing heart disease, and the risk of a heart attack is low; (11-100) a mild amount of plaque is present, e.g., there is mild heart disease with a moderate risk of heart attack (in this case, the doctor may suggest additional treatments alongside lifestyle changes); (101-400) a moderate amount of plaque is present, e.g., the patient has heart disease and the plaque could be obstructing an artery (e.g., the risk of a heart attack is moderate to high and a healthcare provider may recommend further tests and beginning treatment); and (>400) a large amount of plaque is present, e.g., the likelihood that plaque is blocking one of the arteries is over 90%, increasing the already high risk of heart attack (in this case, the healthcare provider will likely order additional tests and initiate treatment).

A study conducted by researchers from the Netherlands found that individuals with CAC scores between 101-500 were at double the risk of mortality compared to a reference group, while individuals with CAC scores >500 had a 2.7 times higher risk of mortality. As high CAC scores are heavily influenced by CAC density, researchers may have previously advocated that a denser CAC may suggest stable calcified plaques, which are less prone to rupture and lower risk of sudden heart attack. However, in the above-mentioned study, it was found that individuals with extremely high CAC scores (e.g., >1000) have a larger CAC area, more extra-coronary calcium, and a significantly higher risk of CVD, coronary heart disease (CHD), cancer, and all-cause mortality compared to individuals with CAC scores between 400 and 999. Moreover, individuals with extremely high CAC scores are at the same or a similar risk level as individuals undergoing secondary prevention. Therefore, individuals with extremely high CAC scores may be have an even greater risk of heart attack, and thus have an immediate need for preventative therapies.

CAC score screening may leverage retinal features linked to calcification scores, providing a unique opportunity to detect cardiovascular risks through retinal imaging. This disclosure integrates retinal images and multi-dimensional data (e.g., sociodemographic data and/or clinical data) to perform CAC screening and optimize cardiovascular risk assessment, particularly where traditional cardiac CT imaging is not feasible. Specifically, this disclosure provides systems and methods for integrating retinal imaging features and multi-dimensional data using a deep learning model tailored to assess coronary artery calcification. By combining fundus image-based biomarkers with a patient's sociodemographic and/or clinical factors, accurate CAC scores and risk levels may be estimated without radiation. Therefore, the systems and methods herein disclose multi-level grading for CAC, from 0 (e.g., no plaque) to over 400 (e.g., high plaque), enhancing preventive care.

Aspects of the disclosure provide various benefits, as outlined below.

Radiation-Free CAC Detection Method Using Retinal Imaging and socio-demographic and clinical data: The disclosure offers a radiation-free, accessible method for CAC detection, utilizing retinal image-derived features and multi-dimensional sociodemographic/medical data. This disclosure introduces a non-invasive, radiation-free CAC scoring method by analyzing retinal images instead of traditional CT scans. Retinal imaging based vascular health status indicators associated with the coronary calcification, allowing for a safer and more accessible form of CAC assessment. The system takes input of retinal images, socio-demographic data and/or clinical data. The images are then processed with normalization, scaling, and/or enhancement for feature extraction. The features are extracted by using advanced deep learning to process these retinal indicators, and then following the feature fusion of the retinal features and socio-demographics and clinical data, the system predicts calcification scores accurately, offering an alternative to radiation-based methods. This innovation reduces patient exposure to harmful imaging while still providing actionable cardiovascular risk insights.

Deep Learning-Based Data Fusion: Aspects of the disclosure employ transformer-based fusion layers which uniquely prioritize data inputs dynamically, and optimize CAC score estimation accuracy. The system employs deep learning-based fusion techniques to integrate retinal image features with socio-demographic and clinical data. A transformer-based model dynamically weighs and prioritizes the most relevant features for accurate CAC score prediction. This data fusion approach allows the model to consider a patient's unique profile, balancing both image and clinical data to improve predictive accuracy. By synchronizing information from multiple sources, the disclosure enhances the model's precision in determining individual CAC scores.

Explainable AI for Cardiovascular Risk Scoring: Aspects of the disclosure employ a unique saliency map-based approach that identifies retinal areas corresponding to calcification indicators, making the model more interpretable and clinically actionable. To promote transparency, the disclosure uses explainable AI techniques, particularly saliency maps, to visually highlight retinal areas that influenced the CAC score prediction. This helps clinicians understand which vascular signs contribute to risk assessment, improving trust and interpretability. The explainable AI feature makes the model's predictions clearer to healthcare providers, supporting informed decision-making and enhancing patient understanding of their cardiovascular risks.

CAC Scoring Without Direct Coronary Imaging: Aspects of the disclosure produce an estimate of CAC score and associated heart disease risk without CT scans, utilizing retinal imaging and sociodemographic factors and/or reducing the need for direct coronary imaging. The disclosure calculates a reliable CAC score without the need for direct coronary imaging, such as CT scans. By examining indirect markers of cardiovascular health, e.g., retinal features and clinical data, the disclosure provides a non-invasive, accessible means of CAC scoring. This approach eliminates the need for expensive and radiation-based imaging equipment, providing healthcare providers with a practical, cost-effective tool for early cardiovascular risk assessment in various settings.

Multi-Level Risk Stratification System for Preventive Care: Aspects of the disclosure employ support multi-tiered CAC risk scoring from 0 to 400+ with corresponding interventions, enabling early detection and preventive care strategies. The disclosure introduces a multi-level risk stratification method that classifies CAC scores into risk categories corresponding to the Agatston scale (0, 1-10, 11-100, 101-400, >400). Each level guides healthcare providers in creating personalized patient management plans, with preventive measures tailored to specific risk levels. This stratification approach helps clinicians make informed choices on early interventions, encouraging patients to take proactive steps based on their precise risk level.

Dynamic Weighting of Sociodemographic and Clinical Data: Aspects of the disclosure employ utilize transformer-based models that dynamically weigh individual risk factors in CAC estimation, enhancing score accuracy for diverse populations. By using transformer-based attention, the disclosure applies dynamic weighting to sociodemographic and clinical inputs, tailoring predictions to each patient's unique risk factors. This allows the model to prioritize factors like age, gender, ethnicity, and medical history according to their relevance to CAC prediction, improving accuracy. This dynamic weighting approach enhances the adaptability and accuracy of CAC predictions across diverse patient populations.

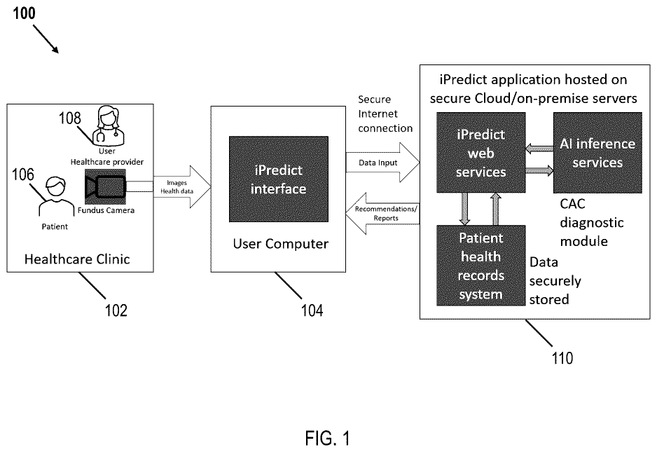

Referring to FIG. 1, there is shown an illustration of an exemplary coronary artery calcification (CAC) estimation system 100 in accordance with aspects of the present disclosure. The CAC estimation system 100 includes a clinic 102 (e.g., a healthcare clinic), a computer system 104 (e.g., a user computer or a mobile device), and/or a prediction system 110 (e.g., an application hosted on a web server).

A user 108 (e.g., a healthcare provider) captures images (e.g., fundus images) and/or health data (e.g., sociodemographic and/or clinical data) related to a patient 106. The images may be captured using a camera, such as a fundus camera (e.g., a mydriatic fundus camera and/or a non-mydriatic fundus camera). In aspects, the camera may be a wide-field fundus camera, smartphone-based fundus camera, adaptive an optical coherence tomography (OCT) camera, scanning laser ophthalmoscope (SLO), multimodal imaging system, and/or other camera configured to capture retinal imaging. The images and/or health data are sent to computer system 104, where they may be displayed via an interface. For example, the images and/or health data may be displayed in an application on a desktop computer or a mobile device through a web interface.

Prediction system 110 includes web services, AI interface services (e.g., a CAC diagnostic module), and/or a records system (e.g., a patient health records system). In aspects, prediction system 110 may be an application hosted on a secure server and/or on-premise server. Computer system 104 and prediction system 110 (e.g., an iPredict system) are configured to exchange data in order to determine a CAC estimation (e.g., a prediction regarding a CAC score). For example, computer system 104 is configured to send data input, such as images and/or health data, to prediction system 110. In turn, prediction system 110 is configured to send recommendations and/or reports to computer system 104, such as predictions related to a CAC score.

The illustrated CAC estimation system 100 is merely exemplary. In aspects, other systems, servers, and/or devices not illustrated in FIG. 1 may be included. In aspects, one or more of the illustrated components may be omitted. Such and other embodiments are contemplated to be within the scope of the present disclosure.

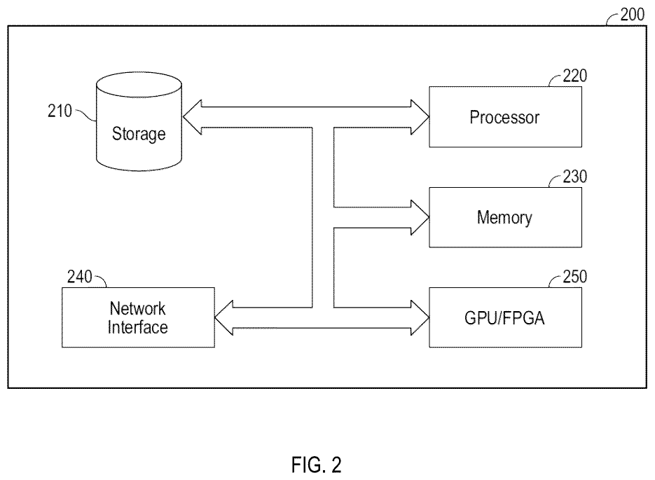

Referring now to FIG. 2, exemplary components of a controller 200 are shown. The controller 200 generally includes a storage or database 210, one or more processors 220, at least one memory 230, and a network interface 240. In aspects, the controller 200 may include a graphical processing unit (GPU) 250, which may be used for processing machine learning network models.

The database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray Disc™, or the like.

In aspects, data may be stored on the controller 200, including, for example, user data, camera data, video streams, and/or other data. The data can be stored in the database 210 and sent via the system bus to the processor 220. The database 210 may store information in a manner that satisfies information security standards and/or government regulations, such as Systems and Organization Controls (e.g., SOC 2), General Data Protection Regulation (GDPR), and/or International Organization for Standardization (ISO) standards.

As will be described in more detail later herein, the processor 220 executes various processes based on instructions that can be stored in the at least one memory 230 and utilizing the data from the database 210. The illustration of FIG. 2 is exemplary, and persons skilled in the art will understand that other components may exist in controller 200. Such other components are not illustrated for clarity of illustration.

Prediction system 110 includes various modules configured to process recommendations and/or reports. Generally, prediction system 110 includes a data collection module 300, a preprocessing module 400, a feature extraction module 500, a data fusion module 600, a risk scoring module 700, an explainability module 800, and/or a patient risk reporting module 900, as discussed further below.

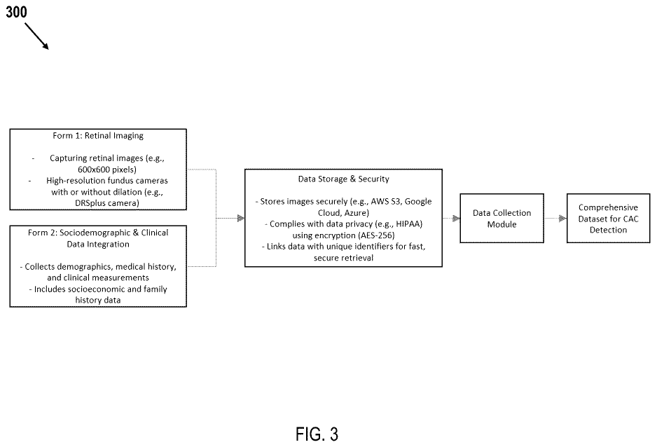

With reference to FIG. 3, there is shown a block diagram of data collection module 300 in accordance with aspects of the present disclosure. Data collection module 300 is configured to collect data from various sources, such as retinal imaging data, sociodemographic data, and/or clinical data for each patient 106, creating a comprehensive dataset for CAC detection.

Retinal imaging data includes retinal images, such as high-resolution retinal fundus images. Typically, retinal imaging data is captured at 600×600 pixels resolution to ensure detailed views of retinal structures such as blood vessels and the optic disc. In aspects, other resolutions (e.g., 512×512 and/or 1024×1024 pixels), may also be used based on model requirements. The retinal imaging data is stored securely in a database, such as database 210. Generally, retinal imaging data is stored in a cloud-based storage service (e.g., AWS S3®, Google Cloud Storage®, and/or Azure Blob Storage®) with encryption options like AES-256 to comply with data privacy laws, including HIPAA.

The retinal fundus images are processed through a neural network model to extract retinal features such as vessel width, bifurcation points, arteriolar narrowing, and/or signs including cotton wool spots and hemorrhage, which correlate with vascular health. Image preprocessing adjusts for noise, lighting, and/or quality consistency, followed by resizing (e.g., to 600×600 pixels) for model compatibility.

Sociodemographic data includes data on patient demographics (e.g., age, gender, and/or ethnicity), medical history (e.g., hypertension, diabetes, and/or smoking status), and/or clinical measurements (e.g., body mass index (BMI) and/or blood pressure). Additional categorical inputs consider socioeconomic indicators and family history of cardiovascular conditions. Clinical data includes patient demographics (e.g., age, gender, and/or ethnicity), health history (e.g., hypertension and/or diabetes), and/or other risk factors (e.g., smoking status, BMI, and/or blood pressure in mmHg).

Example input parameters are outlined below:

Type of input

| parameters | | | Example | Example |
|---|---|---|---|---|
| for CAC | Input Parameters and range | | Patient1 | Patient2 |
| Socio- | Age | 30-85 | 40 | 80 |
| demographic | Race | American | Asian | White |
| and clinical | | Indian or | | |
| parameters | | Alaska Native | | |
| | | Asian | | |
| | | Black or | | |
| | | African | | |
| | | American | | |
| | | Native | | |
| | | Hawaiian or | | |
| | | Other Pacific | | |
| | | Islander | | |
| | | White | | |
| | | Other/multiple | | |
| | Ethnicity | Hispanic or | Not | Not |
| | | Latino | Hispanic | Hispanic |
| | | Not Hispanic | | |
| | | or Latino | | |
| | | No answer | | |
| | Gender | Male | Male | Female |
| | | Female | | |
| | | Other/No | | |
| | | answer | | |
| | Rx for hypertension | Yes/No | No | Yes |
| | History of heart disease | Yes/No | No | Yes |
| | Rx for heart conditions | Yes/No | No | Yes |
| | Diastolic Blood Pressure | mmHg | 80 | 90 |
| | Systolic Blood pressure | mmHg | 120 | 130 |
| | Body mass index | numerical value | 23 | 30 |
| | Smoking Status | Yes/No | No | No |
| | Diabetes | Yes/No | No | No |
| Retinal | Microaneurysm | Severity type | 0 | 1 |
| features | | 0 to 2 | | |
| | Exudates | Severity type | 0 | 1 |
| | | 0 to 2 | | |
| | Hemorrhages | Severity type | 0 | 1 |
| | | 0 to 2 | | |
| | Arteriolar | Severity type | 0 | 1 |
| | Narrowing | 0 to 3 | | |
| | Arteriovenous | Severity type | 0 | 1 |
| | nicking | 0 to 3 | | |

The sociodemographic data and/or the clinical data is stored securely in a database, such as database 210. Generally, sociodemographic data and/or clinical data is stored in structured databases (e.g., PostgreSQL®, MySQL®, and/or NoSQL options like MongoDB® or DynamoDB®) for flexible querying and scalability. Each patient entry is assigned a unique identifier (e.g., UUID or EHR-compatible identifiers) to securely link retinal images with clinical data, ensuring rapid and reliable access across applications. This data structure enables the system to process and retrieve large volumes of patient information with low latency, making it suitable for both research and clinical use.

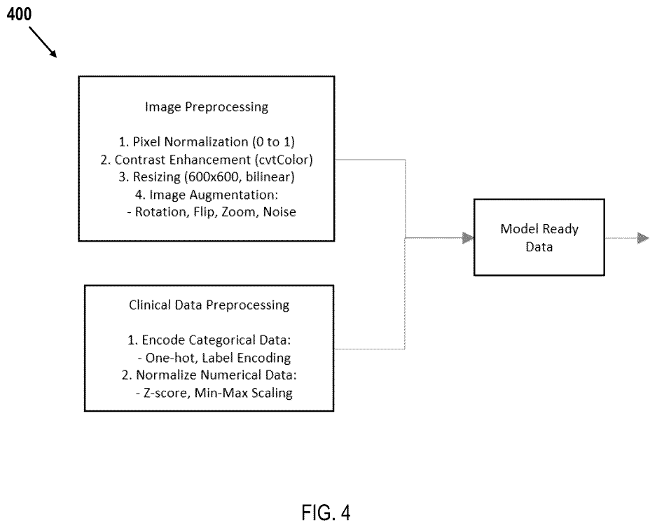

With reference to FIG. 4, there is shown a block diagram of a preprocessing module 400 in accordance with aspects of the present disclosure. The preprocessing module 400 is configured to prepare and/or standardize images and clinical data (e.g., collected by data collection module 300), such as age, gender, ethnicity, blood pressure (in mmHg), BMI, and medical history (e.g., conditions including hypertension, length of hypertension, diabetes, length of diabetes, HDL cholesterol, LDL cholesterol and/or total cholesterol) of patient 106 for model compatibility.

Preprocessing module 400 is configured to perform image preprocessing, which typically includes various steps: pixel normalization, contrast enhancement, resizing, and/or augmentation. This preprocessing pipeline ensures all data conforms to a consistent format for subsequent neural network ingestion, optimizing both training convergence and runtime performance.

Pixel normalization may normalize values in the 0 to 1 range. For example, preprocessing module 400 may apply a normalization function. In aspects, batch normalization may be used to preprocess multiple images. Contrast enhancement may improve visibility of features in an image (e.g., by increasing the difference between lighter and darker regions). For example, preprocessing module 400 may utilize histogram equalization, contrast stretching, gamma correction, unsharp masking, logarithmic transformations, and/or adaptive methods (e.g., machine-learning based methods such as a generative adversarial network (GAN)) on a retinal image of patient 106. Resizing may standardize image sizing for future processing. For example, preprocessing module 400 may resize all fundus images to 600×600 pixels using bilinear interpolation. In aspects, preprocessing module 400 may use applications with high-speed processing to enhance the resizing process. Image augmentation may be performed to increase a sample size. For example, preprocessing module 400 may apply random rotations up to ±15°, horizontal flips, zooming by ±0.1, and/or Gaussian noise with a mean of 0 and/or a standard deviation of 0.05 in order to augment images.

Preprocessing module 400 is further configured to perform clinical data preprocessing, which typically includes encoding categorical data and normalizing numerical data. Encoding categorical data may make data machine-readable, reduce bias, and/or improve model interpretability. For example, preprocessing module 400 may encode data (e.g., gender and/or ethnicity of patient 106) through methods such as one-hot encoding and/or label encoding. Normalizing numerical data may enable models (e.g., machine learning models) to converge faster during training and/or make better predictions. For example, preprocessing module 400 may normalize numerical data (e.g., age, BMI, and/or blood pressure) using z-score normalization, Min-Max scaling, and/or other standardization methods using various tools.

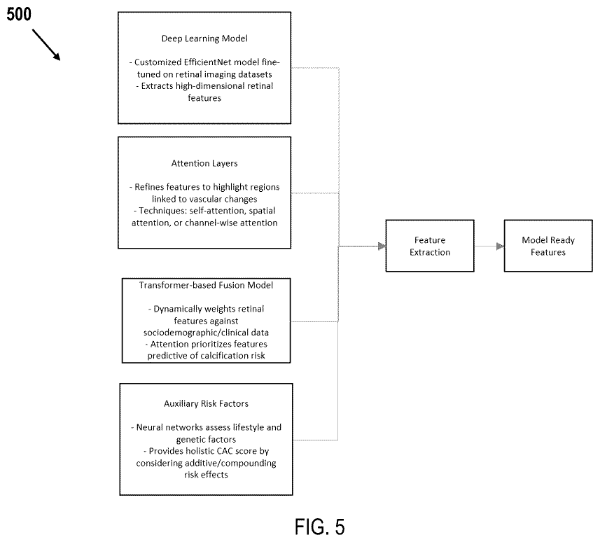

With reference to FIG. 5, there is shown a block diagram of a feature extraction module 500 in accordance with aspects of the present disclosure. Feature extraction module 500 is configured to extract relevant information from data, such as data preprocessed by preprocessing module 400. Generally, feature extraction module 500 utilizes a deep learning model, attention layers, a transformer-based fusion model, and/or one or more auxiliary risk factors.

Feature extraction module 500 is configured to use a machine learning (ML) model, such as a deep learning (DL) model, to identify and/or extract important features from retinal images while focusing on indicators of vascular health linked to calcification. Generally, the machine learning model is pre-trained on large image datasets (e.g., ImageNet), then fine-tuned on specific retinal datasets to highlight retinal features associated with vascular health, including a vessel width, focal arteriolar narrowing, arteriovenous nicking, central arteriolar light reflex, vessel branching patterns, hollenhorst plaque, papilledema, exudates, cotton wool spots, potential hemorrhages, and/or potential microaneurysms. In aspects, feature extraction model 140 may use a convolutional neural network (CNN), (e.g., EfficientNet (B0-B7 variants), ResNet, and/or custom CNN architecture). For example, the ML model may be a customized EfficientNet model fine-tuned on retinal imaging datasets, which is configured to extracts high-dimensional retinal features. Alterative neural network architectures are contemplated and within the scope of this disclosure.

The extracted features may be further refined using an attention layer(s) (e.g., single-head or multi-head attention) to highlight regions associated with vascular changes indicative of calcification risk. For example, the attention layer(s) may further focus on important image regions of the retina, particularly areas including the optic disc, macula, and/or major retinal blood vessels. The attention layer may be implemented using techniques such as self-attention, spatial attention, and/or channel-wise attention, depending on the specific features that are required.

Feature extraction module 500 is further configured to use a transformer-based fusion model, which dynamically weighs retinal image features against sociodemographic and/or clinical data (e.g., while attention layers prioritize features predictive of calcification risk). The model's attention mechanisms may prioritize features most predictive of calcification. For example, for clinical data, dense layers process and embed categorical variables (e.g., gender and/or ethnicity) into dense vector representations, while continuous variables (e.g., age and/or BMI) are passed through additional dense layers to optimize their format for data fusion. This combination of CNNs and dense layers efficiently captures both image-based and numerical/clinical features for comprehensive analysis.

In aspects, auxiliary risk factors may be employed by feature extraction module 500. For example, auxiliary neural networks may assess lifestyle and/or genetic factors of patient 106, providing a holistic CAC score estimation by considering additive or compounding risk effects.

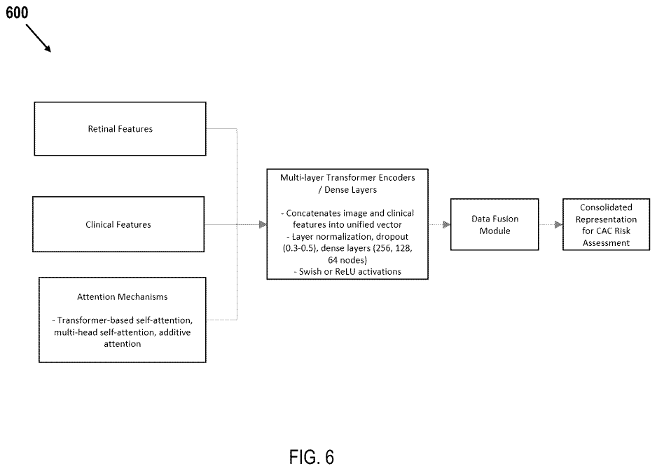

With reference to FIG. 6, there is shown a block diagram of a data fusion module 600 in accordance with aspects of the present disclosure. Data fusion module 600 is configured to integrate (e.g. fuse) extracted retinal and/or clinical features, such as features extracted by feature extraction module 500, thereby allowing a model to analyze data holistically by leveraging both visual and structured data inputs.

Data fusion module 600 achieves data integration through transformer-based self-attention layers, but other attention mechanisms (e.g., multi-head self-attention and/or additive attention) may also be employed to weigh features dynamically based on their relevance to CAC risk prediction. Typically, the image and/or clinical features are concatenated into a unified data vector (e.g., 512-1024 dimensions) and processed through multi-layer transformer encoders and/or traditional dense layers. This configuration applies layer normalization and dropout. For example, the configuration of data fusion module 600 may apply dropout with rates between 0.3-0.5, adjusted to balance generalization and overfitting risk. After attention-based weighting, further dense layers (e.g., 256, 128, 64 nodes) may be employed to refine the combined features to produce a consolidated representation of both visual and clinical data. In aspects, Swish or ReLU activations may be employed, thus enhancing the model's ability to analyze complex relationships between clinical and/or image features, leading to more accurate CAC risk assessments.

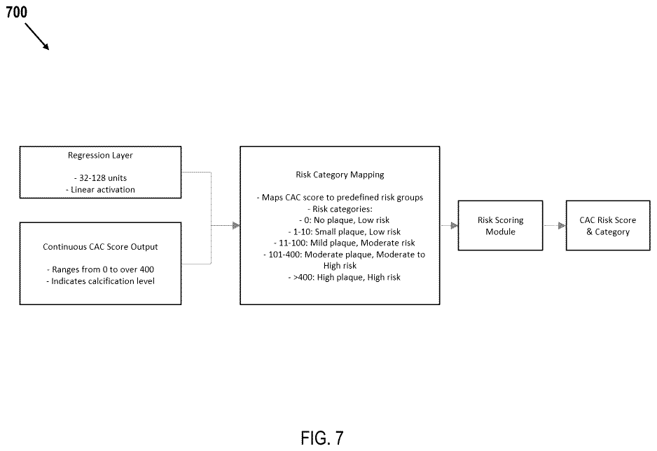

With reference to FIG. 7, there is shown a block diagram of a risk scoring module 700 in accordance with aspects of the present disclosure. Risk scoring module 700 is configured to perform CAC scoring and risk stratification, e.g., based on the integrated data from data fusion module 600.

Risk scoring module 700 is configured to output a continuous CAC score indicative of a calcification level, which may be based on a recognized scale (e.g., an Agatston scale and/or other custom scales). The CAC score may be generated using a regression layer, which produces a continuous value that may range from 0 (e.g., low risk) to over 400 (e.g., high risk) depending on the CAC level. The regression layer may have between 32 and 128 units depending on the model configuration and is typically followed by a single neuron output with linear activation to predict a continuous CAC score.

This score is then mapped to risk categories. For example, risk scoring module 700 may map a CAC score to a predefined risk group and/or category, such as: (0) No plaque, e.g., low risk; (1-10) small plaque, e.g., low risk; (11-100) mild plaque, e.g., moderate risk; (101-400) moderate plaque, e.g., moderate to high risk; and (>400) high plaque amount, e.g., high risk. In aspects, the scores (e.g., thresholds) may be aligned with established cardiovascular risk profiles, which can be further customized based on clinical preferences and/or dataset-specific adjustments. The model training process typically minimizes mean squared error (MSE) for continuous score optimization, with an option to incorporate categorical cross-entropy loss if classifications are validated against known risk thresholds, making the risk scoring module 700 adaptable to various clinical applications.

The final layer of the neural network outputs a continuous CAC score prediction, which is generally mapping onto the Agatston score scale. For classification, the score may be segmented into risk groups based on predefined ranges. For example, the score may be segmented into: (0) no plaque with low risk; (1-10) low plaque with less than a 10% chance of heart disease; (11-100) mild plaque with moderate heart disease risk; (101-400) moderate plaque, suggesting heart disease with moderate-high risk; and (>400) high plaque with over 90% likelihood of artery blockage and/or high heart attack risk.

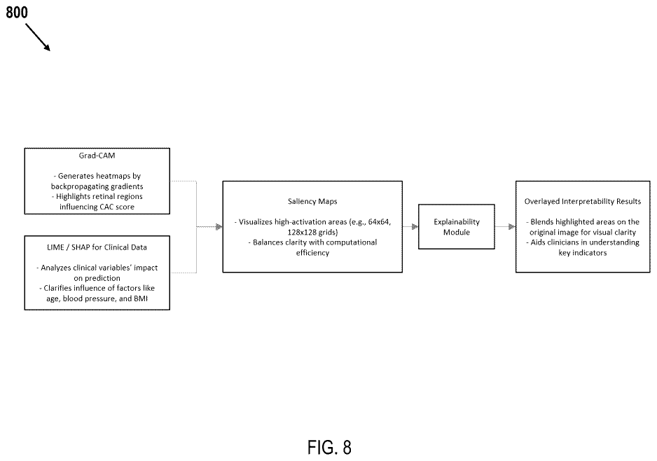

With reference to FIG. 8, there is shown a block diagram of an explainability module 800 in accordance with aspects of the present disclosure. Explainability module 800 is configured to provide clinicians (e.g., healthcare provider 108) with a visual interpretation (e.g., a textual and/or visual explanation) of model predictions (e.g., scores), thereby highlighting specific retinal regions (e.g., of the retina of patient 106) that contributed to the CAC score generated by risk scoring module 700.

Explainability module 800 may utilize grad-CAM (gradient-weighted class activation mapping), LIME (local interpretable model-agnostic explanations), and/or other explainability tools (e.g., SHAP values for clinical data). Grad-CAM is configured to generate heatmaps by backpropagating gradients from the output layer to the earlier convolutional layers, highlighting areas such as vascular regions and/or the optic disc based on their influence on the model's prediction. Saliency maps, which visualize these areas with high activation, are created with flexible grid sizes (e.g., 64×64, 128×128) to balance computational efficiency and visual clarity. For further clinical interpretability, LIME and/or SHAP values can be used to analyze clinical data, clarifying the importance of variables like age, blood pressure, and/or BMI on the CAC prediction. The final interpretability results are overlaid on the original image (e.g., retinal image of patient 106). In aspects. The final results use alpha blending for clear contrast, aiding healthcare provider 108 in understanding key indicators behind each risk score.

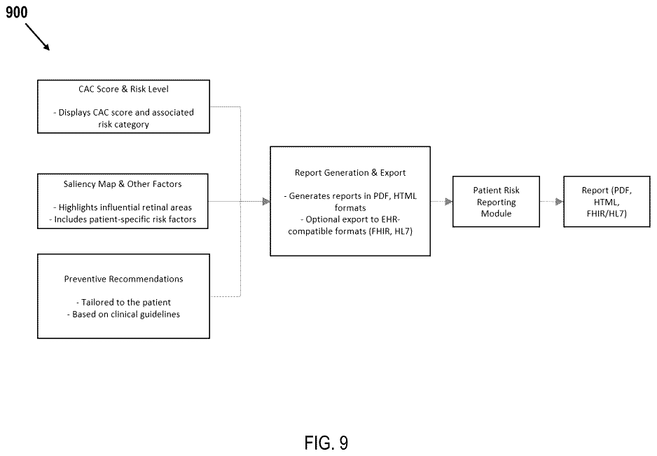

With reference to FIG. 9, there is shown a block diagram of a patient risk reporting module 900 in accordance with aspects of the present disclosure. Patient risk reporting module 900 is configured to compile output, including CAC scores, risk levels, and/or visual explanations, into a cohesive report for clinical use.

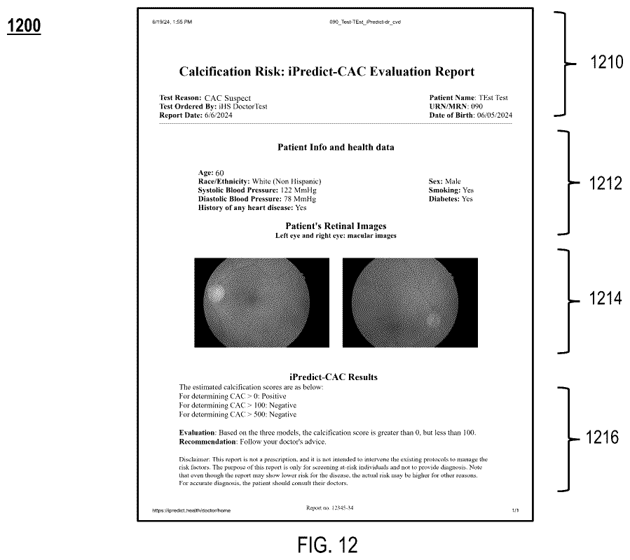

The final output (e.g., report 1200) (FIG. 12) is generated dynamically, often through a web-based interface, which allows real-time access and interactivity. Each report typically includes: (1) CAC Score with its associated risk level; (2) Saliency Map, displaying influential retinal areas, and other risk factors for the patients; and (3) Preventive Recommendations. The report 1200 is typically based on clinical guidelines and/or personalized for the patient. Reports are saved in portable formats such as a portable document format (PDF) or hypertext markup language (HTML) using various libraries or other PDF generation tools for ease of sharing and/or secure storage. In aspects, patient risk reporting module 900 is configured to optionally export data in standardized formats (e.g., health level seven (HL7) or FHIR JSON) to enhance integration with healthcare systems, making the system 100 and/or output (e.g., a report 1200) compatible with electronic health record (EHR) systems and/or facilitating long-term patient tracking. The report 1200 provides the benefits of a complete, interpretable, and/or actionable summary, supporting clinician decision-making and patient education that improves the healthcare system and the overall accuracy and completeness of CAC scoring for patient diagnosis.

In aspects, the information (e.g., CAC estimation), evaluations, and/or recommendations provided in report 1200 may be output in various formats. For example, the report 1200 may be output to a display screen of computer system 104 via a local application or web-based application. The report 1200 may be in the form of an alert, such as an SMS text, phone call, and/or push alert. The report 1200 may include visual, audio, haptic, and/or additional information demonstrating the CAC estimation.

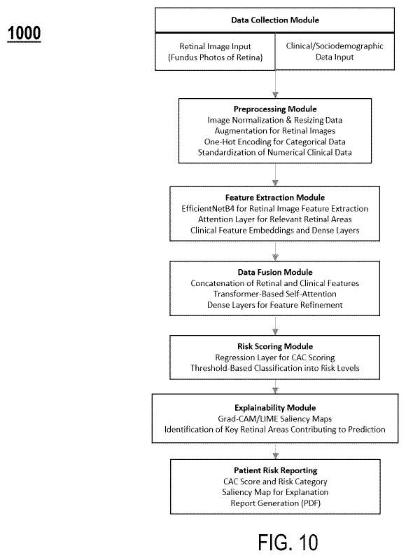

With reference to FIG. 10, there is shown a block diagram illustrating data flow within the CAC estimation system 100, in accordance with aspects of the present disclosure. For example, the data may first be collected through data collection module 300, then processed through various additional modules until reaching the patient risk reporting module 900, which outputs the final report 1200.

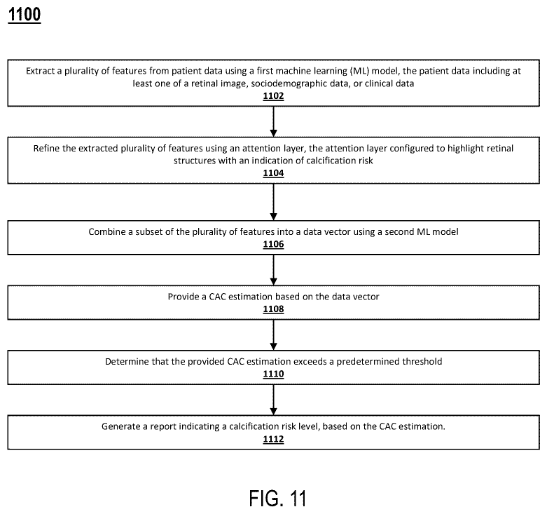

With reference to FIG. 11, there is shown a method 1100 for an exemplary use of the CAC estimation system 100. Although the steps of method 1100 of FIG. 11 are shown in a particular order, the steps need not all be performed in the specified order, and certain steps can be performed in another order. For example, FIG. 11 will be described below, with a server (e.g., controller 200 of FIG. 2) performing the operations. In various aspects, the method 1100 of FIG. 11 may be performed all or in part by controller 200. In other aspects, the method 1100 of FIG. 11 may be performed all or in part by another device, for example, a mobile device and/or a client computer system. These and other variations are contemplated to be within the scope of the present disclosure.

Initially, at step 1102, the controller 200 causes CAC estimation system 100 to extract a plurality of features from patient data using a first ML model. The patient data may include a retinal image, sociodemographic data, and/or clinical data

For example, data collection module 300 may initially capture and/or receive retinal images of patient 106, such as fundus photos of a retina, and/or information regarding the patient's age and medical history. In aspects, the patient data may be fed to preprocessing module 400 for image normalization and/or augmentation, such as encoding of categorical data and/or standardization of numerical clinical data. Next, feature extraction module 500 is configured to extract features from the patient data, such as relevant retinal areas and clinical feature embeddings and/or dense layers.

Next, at step 1104, the controller 200 causes CAC estimation system 100 to refine the extracted plurality of features using an attention layer. The attention layer is generally configured to highlight retinal structures with an indication of calcification risk. For example, data fusion module 600 may concatenate retinal and clinical features using transformer-based self-attention and/or dense layers or data refinement.

Next, at step 1106, the controller 200 causes CAC estimation system 100 to combine a subset of the plurality of features into a data vector using a second ML model. For example, data fusion module 600 may cause the image and/or clinical features to be concatenated into a unified data vector.

Next, at step 1108, the controller 200 causes CAC estimation system 100 to provide a CAC estimation based on the data vector. For example, risk scoring module 700 may use regression layers for CAC scoring. The Agatston scoring model is typically used.

Next, at step 1110, the controller 200 causes CAC estimation system 100 to determine that the provided CAC estimation exceeds a predetermined threshold. For example, risk scoring module 700 may use threshold-based classification to produce risk levels, e.g., risk of high calcification in vessels. The explainability module 800 may be used to interpret and/or explain the risk levels.

Next, at step 1112, the controller 200 causes CAC estimation system 100 to generate an output indicating a calcification risk level based on the CAC estimation. For example, the information generated by explainability module 800 may be concatenated into a report 1200, which is generated by patient risk reporting module 900.

With reference to FIG. 12, there is shown an exemplary illustration of a report 1200 generated by the CAC estimation system 100, in accordance with aspects of the present disclosure. The report 1200 may include sections illustrating and explaining the test results.

A first section 1210 of report 1200 may include patient 106 and healthcare provider 108 information. For example, the first section 1210 may include a test reason (e.g., “CAC suspect”), the healthcare provider 108 that ordered the test, the report date (e.g., Jun. 6, 2024), the patient's name, a uniform resource name (URN) and/or medical record number (MRN) (e.g., “090”), and/or the patient's date of birth (e.g., Jun. 5, 2024). A second section 1212 may include information and/or health data related to the patient 106. For example, the second section 1212 may include a patient's age, sex, race/ethnicity, systolic blood pressure (e.g., 122 MmHg), diastolic blood pressure (e.g., 78 MmHg), and/or history of heart disease, smoking, and/or diabetes.

A third section 1214 of report 1200 may include retinal images of patient 106, e.g., macular images of the left eye and right eye. A fourth section of report 1216 may include CAC predictions (e.g., test results). For example, the CAC results may state estimated calcification scores, such as “For determining CAC>0: Positive; For determining CAC>100: Negative; For determining CAC>500: Negative”. In addition, the fourth section 1216 may include an evaluation and/or recommendation. For example, an evaluation may interpret and/or explain the test results, stating “Based on the three models, the calcification score is greater than 0, but less than 100.” In another example, a recommendation may provide treatment instructions, such as “Follow your doctor's advice” or “Please seek a follow-up visit in 2-3 weeks”. In aspects, the evaluation and/or recommendation may be generated using a ML model.

System 100 refers to various modules and/or ML models. It will be understood that various ML architecture alternatives, such as neural network architectures, may be employed by system 100. For example, system 100 may use a ResNet (residual network), which enables residual connections that help in training deep networks without vanishing gradients and/or are beneficial for extracting detailed features from high-resolution images. In another example, system 100 may use a DenseNet (dense convolutional network), which enables dense connections between layers, improving information flow and enabling feature reuse, which useful for capturing fine retinal details. In another example, system 100 may use VGG (visual geometry group networks) with deep layers, which enable simplicity and efficiency, especially in feature extraction for smaller datasets. In still another example, system 100 may use RegNet (regularization networks), which are flexibly designed for efficient scaling and regularization, allowing for better feature extraction with customizable architecture options.

Additional architectures for ML may include: multi-scale convolutions (e.g., to capture different levels of detail in images suitable for complex patterns in retinal images), depth-wise separable convolutions (e.g., ideal for fast processing in edge devices), compact architectures that achieves high-level accuracy with 50× fewer parameters (e.g., suitable for limited computing environments), replacing standard convolutions with depth wise separable convolutions (e.g., achieving high efficiency and detail extraction), Auto ML-generated architecture optimized for high performance with minimal computational cost (e.g., useful in high-dimensional medical imaging), scalable and efficient models balancing accuracy and/or efficiency (e.g., by scaling depth, width, and resolution), simple and relatively shallow networks (e.g., a good option for baseline models, especially when computational resources are limited), lightweight and optimized models for mobile devices (e.g., using pointwise group convolutions, suitable for real-time applications on constrained hardware), transformer-based architectures that process image patches as sequences, (e.g., capable of capturing long-range dependencies useful for more global image interpretations), adaptations of convolutional models including design choices from transformers (e.g., achieving competitive results in vision tasks), and architectures specifically designed for medical imaging with segmentation and/or adapted for feature extraction (e.g., encoder-decoder structure capturing spatial information). It is understood that alternative network architectures are contemplated and within the scope of this disclosure.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi®, Fortran®, Java®, JavaScript®, machine code, operating system command languages, Pascal®, Perl®, PL1, scripting languages, Visual Basic, metalanguages which themselves specify programs, and all first, second, third, fourth, fifth, or further generation computer languages. Also included are database and other data schemas, and any other meta-languages. No distinction is made between languages that are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above are also intended to be within the scope of the disclosure.

1. (canceled)

2. The system of claim 21, wherein the first ML model is a deep learning model.

3. The system of claim 21, wherein the sociodemographic data includes at least one of an age, gender, or race.

4. The system of claim 21, wherein the clinical data includes at least one of a presence of hypertension, cholesterol, blood pressure, body mass index (BMI), smoking status, or a presence of diabetes.

5. The system of claim 21, wherein the instructions, when executed by the processor, further cause the system to:
preprocess the patient data using pixel normalization, contrast enhancement, or image scaling.

6. The system of claim 21, wherein the instructions, when executed by the processor, further cause the system to:
preprocess the patient data using label encoding or one-hot encoding.

7. The system of claim 21, wherein the plurality of features extracted from the patient data include at least one of a vessel width, focal arteriolar narrowing, arteriovenous nicking, central arteriolar light reflex, vessel branching patterns, hollenhorst plaque, papilledema, exudates, cotton wool spots, potential hemorrhages, and/or potential microaneurysms.

8. The system of claim 21, wherein the highlighted retinal structures include at least one of an optic disc, a macula, or a retinal blood vessel.

9. The system of claim 21, wherein the attention layer is implemented using at least one of self-attention, spatial attention, or channel-wise attention.

10. The system of claim 21, wherein the second ML model is a transformer-based fusion model configured to dynamically weigh the extracted plurality of features.

11. (canceled)

12. The method of claim 24, wherein the first ML model is a deep learning model.

13. The method of claim 24, wherein the sociodemographic data includes at least one of an age, gender, or race.

14. The method of claim 24, wherein the clinical data includes at least one of a presence of hypertension, cholesterol, blood pressure, body mass index (BMI), smoking status, or a presence of diabetes.

15. The method of claim 24, further comprising to:
preprocessing the patient data using pixel normalization, contrast enhancement, or image scaling.

16. The method of claim 24, further comprising:
preprocessing the patient data using label encoding or one-hot encoding.

17. The method of claim 24, wherein the plurality of features extracted from the patient data include at least one of a vessel width, focal arteriolar narrowing, arteriovenous nicking, central arteriolar light reflex, vessel branching patterns, hollenhorst plaque, papilledema, exudates, cotton wool spots, potential hemorrhages, and/or potential microaneurysms.

18. The method of claim 24, wherein the highlighted retinal structures include at least one of an optic disc, a macula, or a retinal blood vessel.

19. The method of claim 24, wherein the attention layer is implemented using at least one of self-attention, spatial attention, or channel-wise attention.

20. (canceled)

21. A system for coronary artery calcium (CAC) estimation, comprising:
a fundus imaging interface configured to receive at least one color retinal fundus image captured by a fundus camera;
a processor; and
a memory storing instructions that, when executed by the processor, cause the system to:
perform image-quality gating on the retinal fundus image by computing at least one of focus sharpness, illumination uniformity, or vessel-to-background contrast and rejecting images below a threshold;
preprocess the accepted retinal fundus image by pixel normalization and contrast enhancement and generate a vessel-probability map by retinal vessel segmentation;
extract a plurality of features from patient data using a first machine learning (ML) model, the patient data including at least one of a retinal fundus image, sociodemographic data, or clinical data;
refine the extracted plurality of features using an attention layer, the attention layer trained to and configured to (i) highlight retinal structures indicative of calcification risk using localization targets corresponding to optic disc, macula, and vessel masks;
combine a subset of the plurality of features and the encoded sociodemographic and clinical embeddings into a data vector using a second ML model comprising a transformer encoder with multi-head self-attention and positional encodings;
provide a CAC estimation as a continuous score aligned to an Agatston scale based on the data vector using a regression head trained with CT-derived CAC ground truth;
calibrate the continuous CAC score by isotonic regression or Platt scaling on a held-out validation set;
determine that the provided calibrated CAC score exceeds a predetermined threshold corresponding to a risk category; and
generate an output indicating a calcification risk level, based on the calibrated CAC score.

22. The system of claim 21, wherein the output comprises a structured clinician report that includes (i) the risk category and (ii) an overlay saliency map highlighting image regions contributing to the estimation.

23. The system of claim 21, wherein the report is exported in HL7 or FHIR format.

24. A processor-implemented method for coronary artery calcium (CAC) estimation, the method comprising:
extracting a plurality of features from patient data using a first machine learning (ML) model, the patient data including at least one of a retinal fundus image, sociodemographic data, or clinical data;
performing image-quality gating on the fundus image and retinal vessel segmentation to generate a vessel-probability map;
refining the extracted plurality of features using an attention layer, the attention layer configured to highlight retinal structures indicative of calcification risk using supervised attention constrained by optic disc, macula, and vessel masks;
combining a subset of the plurality of features and encoded sociodemographic/clinical embeddings into a data vector using a second ML model comprising a transformer encoder;
providing a CAC estimation as a continuous Agatston-aligned score based on the data vector and calibrating the score by isotonic regression or Platt scaling;
determining that the provided calibrated CAC score exceeds a predetermined threshold; and
generating an output indicating a calcification risk level, based on the calibrated CAC score.

25. The method of claim 24, wherein the step of generating an output includes rendering a clinician-facing report with an overlay saliency map and exporting the report in HL7 or FHIR format.

26. A non-transitory computer readable storage medium including instructions that, when executed by a computer, cause the computer to perform a method for coronary artery calcium (CAC) estimation, the method comprising:
extracting a plurality of features from patient data using a first machine learning (ML) model, the patient data including at least one of a retinal fundus image, sociodemographic data, or clinical data;
performing image-quality gating on the fundus image and retinal vessel segmentation to generate a vessel-probability map;
refining the extracted plurality of features using an attention layer, the attention layer configured to highlight retinal structures indicative of calcification risk using supervised attention constrained by optic disc, macula, and vessel masks;
combining a subset of the plurality of features and encoded sociodemographic/clinical embeddings into a data vector using a second ML model comprising a transformer encoder;
providing a CAC estimation as a continuous Agatston-aligned score based on the data vector and calibrating the score by isotonic regression or Platt scaling;
determining that the provided calibrated CAC score exceeds a predetermined threshold; and
generating an output indicating a calcification risk level, based on the calibrated CAC score.

27. The non-transitory computer readable storage medium of claim 24, wherein the step of generating an output includes rendering a clinician-facing report with an overlay saliency map and exporting the report in HL7 or FHIR format.
